(19) United States
(12) Patent Application Publication
(10) Pub. No.: US 2014/0350506 A1
(43) Pub. Date: Nov. 27, 2014

(54) Diaper collector bag

(71) Applicant: Eric Ponieman, Albertson, NY (US)

(72) Inventor: Eric Ponieman, Albertson, NY (US)

(21) Appl. No.: 13/902,904

(22) Filed: May 27, 2013

(51) Int. Cl. A61F 13/495

(52) U.S. Cl. CPC A61F 13/495
USPC 604/385.19

(57) ABSTRACT

A diaper and method for using the diaper are provided. The diaper includes an impermeable outer layer; an absorbent inner layer; and a collector bag including an opening and an adhesive surrounding the opening; the collector bag being removably attached to the absorbent inner layer.

### BACKGROUND OF THE INVENTION

1. Field of the Invention

The present invention relates generally to a diaper, and more particularly, to a diaper including a collector bag.

2. Description of the Related Art

In a conventional diaper, when a user defecates in a diaper, the user's fecal matter may leave the diaper area and contact the user's skin and/or clothing. This leads to contact with the fecal matter by a caregiver and requires numerous cleaning wipes to clean the soiled area(s). In addition, because the fecal matter carries bacteria, the bacteria is spread to every area that comes into contact with the fecal matter.

### SUMMARY OF THE INVENTION

The present invention has been made to address at least the disadvantages described above and to provide at least the advantages described below.

Accordingly, aspects of the present invention provide a diaper and method for using the diaper for limiting the contact of fecal matter by a user's body and by a caregiver.

According to an aspect of the present invention, there is provided a diaper, comprising an impermeable outer layer; an absorbent inner layer; and a collector bag including an opening and an adhesive surrounding the opening; the collector bag being removably attached to the absorbent inner layer.

According to another aspect of the present invention, there is provided a method of using a diaper, comprising: removably attaching a collector bag to an absorbent inner layer of the diaper, the collector bag including an opening and an adhesive surrounding the opening; removably attaching the collector bag and the diaper to a user.

### BRIEF DESCRIPTION OF THE DRAWINGS

The above and other aspects, features and advantages of certain embodiments of the present invention will be more apparent from the following detailed description taken in conjunction with the accompanying drawings, in which:

FIG. 1 illustrates a diaper, according to an embodiment of the present invention; and

FIG. 2 is a rear view of the diaper as worn by a user, according to an embodiment of the present invention.

### DETAILED DESCRIPTION OF EMBODIMENTS OF THE PRESENT INVENTION

Hereinafter, embodiments of the present invention are described in detail with reference to the accompanying drawings. Detailed descriptions of well-known functions and structures incorporated herein will be omitted to avoid obscuring the subject matter of the present invention. The term “and/or” used herein includes any and all combinations of one or more of the associated listed items.

The terminology used herein is for the purpose of describing embodiments only and is not intended to be limiting. As used herein, the singular forms are intended to include plural forms as well, unless the context clearly indicates otherwise. It will be further understood that the terms “comprises” and/or “has” when used in this description, specify the presence of stated feature, number, step, operation, component, element, or a combination thereof but do not preclude the presence or addition of one or more other features, numbers, steps, operations, components, elements, or combinations thereof.

The terms used herein, including technical terms, have the same meanings as terms that are generally understood by those skilled in the art, as long as the terms are not explicitly defined differently. It should be understood that terms defined in a commonly used dictionary have meanings coinciding with those of terms in the related technology. As long as the terms are not defined obviously, they are not ideally or excessively analyzed as formal meanings

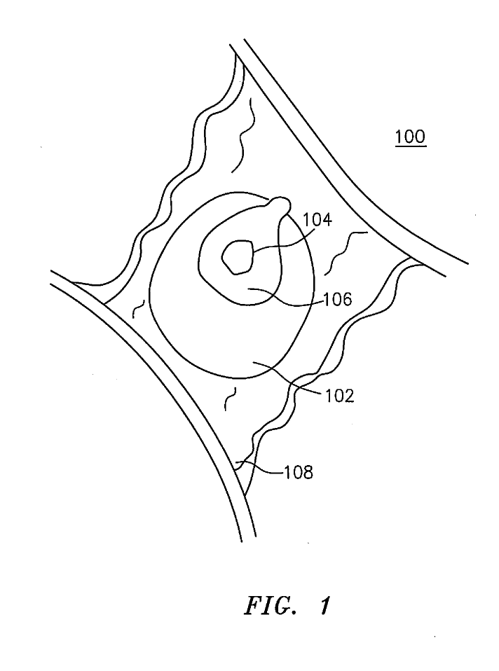

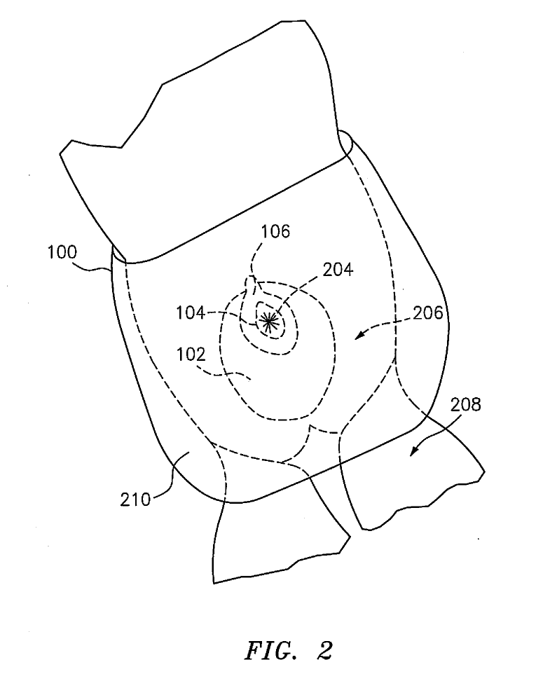

FIG. 1 illustrates a diaper 100, according to an embodiment of the present invention. The diaper 100 may include any conventional diaper. For example, the diaper 100 may be a reusable diaper or a disposable diaper. The diaper 100 includes a collector bag 102, an absorbent inner layer 108, and an impermeable outer layer 210 (FIG. 2). The absorbent inner layer 108 includes any material that is capable of absorbing fluids/liquids. For example, if the user urinates while wearing the diaper 100, the absorbent inner layer 108 absorbs the urine to prevent it from spreading to other areas covered by the diaper 100 and areas outside of the diaper 100. The impermeable outer layer 210 prevents the collected urine from leaking outside of the diaper 100.

The collector bag 102 is formed of any conventional material capable of holding and containing fecal matter in the interior of the collector bag. For example, the collector bag 102 may be formed of plastic. However, it should be noted that any material capable of containing the fecal matter inside of the collector bag 102 may be used. The collector bag 102 is removably attached to the absorbent inner layer 108 using any conventional attachment means such as, for example, double-sided tape, VELCRO®, etc. The collector bag 102 includes an opening 104 that leads into the interior of the collector bag 102. As will be described below, the opening 104 is positioned adjacent to a user's anal orifice so that the user's fecal matter is directed into the interior of the collector bag 102. The collector bag 102 includes an adhesive portion 106 that substantially surrounds the opening 104. The adhesive portion 106 is used to correctly position the collector bag 102 so that the opening 104 is aligned with the user's anal orifice. The adhesive portion 106 may be formed of any conventional material that exhibits adhesive properties such as, for example, double-sided tape.

FIG. 2 illustrates a rear view of the diaper 100 as worn by a user. In the description of FIG. 2, elements described with respect to FIG. 1 will be referred to using the same reference characters as in FIG. 1. Referring to FIG. 2, the diaper 100 is worn by the user and the collector bag 102 is positioned adjacent to the user's buttocks 206 so that the opening 104 is aligned with the user's anal orifice 204. When the user defecates, the fecal matter enters the interior of the collector bag 102 through the opening 104. Accordingly, the fecal matter is contained within the bag and is prevented from spreading to other anatomical areas within the diaper 100 as well as areas outside of the diaper 100, such as, for example, the user's leg 208. As a result, infections such as, for example, skin infections and urinary tract infections are avoided.

In user, the collector bag 102 is replaced with a new collector bag when it becomes full. Because the fecal matter is contained inside of the collector bag 102, the diaper 100 may be reused with the new collector bag unless the absorbent inner layer 108 contains urine. Reusing the diaper 100 and replacing only the collector bag 102 is significantly more cost efficient than replacing the diaper 100 every time the user defecates.

While the present invention has been particularly illustrated and described with reference to certain embodiments thereof, various modifications or changes can be made without departing from the scope of the present invention. Therefore, the scope of the present invention is not limited to the described embodiments, and should be defined by the scope of the following claims and any equivalents thereof.

1-4. (canceled)

5. A diaper, comprising:
an impermeable outer layer;
an absorbent inner layer; and
a collector bag including an opening and an adhesive surrounding the opening; the collector bag being removably attached to the absorbent inner layer.

6. The diaper of claim 5, wherein the opening leads into an interior of the collector bag.

7. The diaper of claim 6, wherein the adhesive aligns the opening with an anal orifice of a user.

8. The diaper of claim 5, wherein the collector bag prevents fecal matter inside of the collector bag from contacting the absorbent layer and escaping the diaper.

9. A method of using a diaper, comprising:
removably attaching a collector bag to an absorbent inner layer of the diaper, the collector bag including an opening and an adhesive surrounding the opening;
removably attaching the collector bag and the diaper to a user.

10. The method of claim 9, wherein the opening leads into an interior of the collector bag.

11. The method of claim 10, wherein the adhesive aligns the opening with an anal orifice of a user.

12. The method of claim 9, wherein the collector bag prevents fecal matter inside of the collector bag from contacting the absorbent layer and escaping the diaper.
